(19) United States
(12) Patent Application Publication
(10) Pub. No.: US 2010/0092581 A1
(43) Pub. Date: Apr. 15, 2010

(54) ANTIBACTERIAL COMPOSITIONS COMPRISING AN EXTRACT FROM ARCEUTHOBIUM

(76) Inventors: Cynthia Ross Friedman, Kamloops (CA); Kathryn Yvonne Pernitsky, Kamloops (CA)

Correspondence Address: BERLINER & ASSOCIATES, 555 WEST FIFTH STREET, 31ST FLOOR, LOS ANGELES, CA, 90013, US

(21) Appl. No.: 12/596,684

(22) PCT Filed: Apr. 18, 2008
(86) PCT No.: PCT/CA08/00731

§ 371 Date: Oct. 20, 2009

Related U.S. Application Data
(60) Provisional application No. 60/913,222, filed on Apr. 20, 2007.

(51) Int. Cl. A61K 36/185; A61P 31/04

(52) U.S. Cl. 424/725

(57) ABSTRACT

This application relates to an antibacterial composition comprising an extract from a plant belonging to the genus Arceuthobium, such as Arceuthobium americanum. The composition may be used in a pharmaceutical composition or a disinfectant. The invention also relates to a method of treating or preventing a bacterial infection, the method comprising administering to a patient a composition comprising the extract.

### RELATED APPLICATIONS

This application claims priority to U.S. Provisional Patent Application No. 60/913,222, filed 20 Apr. 2007 and entitled “Compositions Comprising An Extract From Arceuthobium”, which is incorporated herein by reference.

### TECHNICAL FIELD

This application relates to antibiotic compositions comprising a plant extract. This application also relates to methods of treating or preventing bacterial infections by administering compositions comprising the plant extract.

### BACKGROUND

Antibiotics are used to kill or inhibit the growth of infectious organisms such as pathogenic bacteria. Unfortunately, the increased use of antibiotics has been accompanied by an alarming spread of antibiotic-resistant bacteria. Methicillin-resistant strains of Staphylococcus aureus, for example, are now a common cause of infections in hospitals throughout the industrialized countries. Because of the increase in drug resistance among pathogenic bacteria, there is a need for the development of novel antibiotics, especially those exhibiting efficacy against drug-resistant bacteria.

### SUMMARY OF INVENTION

In accordance with the invention, an antibiotic composition comprising an extract from a plant belonging to the genus Arceuthobium, such as Arceuthobium americanum is disclosed. The composition may be used in a pharmaceutical composition or a disinfectant. The invention also relates to a method of treating or preventing a bacterial infection, the method comprising administering to a patient a composition comprising the extract.

### BRIEF DESCRIPTION OF DRAWINGS

In drawings which illustrate embodiments of the invention, but which should not be construed as restricting the spirit or scope of the invention in any way:

FIG. 1 is a photograph showing the effect of “raw” extracts of Arceuthobium americanum against the growth of Stapyhlococcus aureus using a modified Kirby Bauer Disc Diffusion Method;

FIG. 2 is a photograph showing the effect of “raw” extracts of Arceuthobium americanum against the growth of methicillin-resistant Stapyhlococcus aureus using a modified Kirby Bauer Disc Diffusion Method;

FIG. 3 is a graph showing the effect of “raw” extracts of Arceuthobium americanum (male shoots harvested in May) on the growth of Stapyhlococcus aureus measured by optical density;

FIG. 4 is a graph showing the effect of “raw” extracts of Arceuthobium americanum (male shoots harvested in August) on the growth of Stapyhlococcus aureus measured by optical density;

FIG. 5 is a graph showing the effect of “raw” extracts of Arceuthobium americanum (male shoots harvested in May) on the growth of methicillin-resistant Stapyhlococcus aureus measured by optical density; and

FIG. 6 is a graph showing the effect of “raw” extracts of Arceuthobium americanum (male shoots harvested in August) on the growth of methicillin-resistant Stapyhlococcus aureus measured by optical density.

FIG. 7 is a photograph showing the effect of “6:4” extracts of Arceuthobium americanum against the growth of (a) Stapyhlococcus aureus and (b) methicillin-resistant Stapyhlococcus aureus using a modified Kirby Bauer Disc Diffusion Method.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This application relates to novel antibiotic compositions comprising extracts from plants belonging to the genus Arceuthobium, and to novel methods for treating or preventing bacterial infections using such compositions.

The genus Arceuthobium, commonly known as the dwarf mistletoe, comprises approximately 40 species of parasitic flowering plants which grow in the Northern hemisphere. Each species grows on one to several specific host trees of the Pinaceae (pine) and Cupressaceae (cedar) families. Arceuthobium americanum, for example, grows in western North America and is found primarily as a parasite on the lodgepole pine (Pinus contorta) and jack pine (Pinus banksiana). Arceuthobium plants are evergreen and remain on their hosts throughout the year. All Arceuthobium species are dioecious, having male and female reproductive portions on separate plants. Arceuthobium seeds are discharged in the fall and do not germinate until the following spring.

The present inventors have discovered that extracts of Arceuthobium plants have potent antimicrobial activity.

One aspect of the present invention relates to an antimicrobial composition comprising an extract of Arceuthobium. The extract may be obtained from plant material by extraction with an organic solvent. The plant material may be shoots of Arceuthobium americanum, for example. The solvent may be selected, for example, from the group consisting of methanol, ethanol, chloroform and ethyl acetate. The extract is then concentrated by evaporation until thickened. The concentrated extract is diluted with a small volume of organic solvent and then agitated, for example, by sonication. The solvent is evaporated and any remaining water is removed, for example by vacuum drying, to leave a dry extract. An antimicrobial composition according to one embodiment of the present invention is comprised of this extract.

The novel compositions of the invention are useful as antimicrobials. The compositions are useful, for example, as pharmaceutical compositions. The pharmaceutical compositions may be used to treat or prevent various bacterial infections including infections caused by gram-positive bacteria such as Staphylcoccus aureus and antibiotic-resistant bacteria such as methicillin-resistant Staphylcoccus aureus.

The pharmaceutical compositions of the invention may include a pharmaceutically acceptable diluent, carrier or adjuvant. The compositions may be in the form of a capsule, tablet, powder, liquid or gel, for example. The compositions may be administered to a patient by way of oral, nasal, ophthal, rectal, vaginal, topical, intravenous, subcutaneous, or parenteral administration, for example. The compositions may be useful in human and veterinary applications.

The compositions are useful as surface disinfectants. The disinfectant compositions may be in the form of aqueous or non-aqueous detergent solutions or sprays, for example. These compositions may be useful in medical and veterinary facilities, for example, in disinfecting medical and dental equipment.

Other aspects and advantages of the invention will become apparent from the following non-limiting examples which illustrate the invention.

### Example 1

### Sample Collection

Male and female Arceuthobium americanum shoots were collected in May and August. All samples were taken from lodgepole pines in the Stake Lake area near Kamloops, British Columbia, Canada (latitude 50° 31′, longitude 120° 28′). The shoots consisting of the entire reproductive portion of the plant (the part of the plant growing outside of the tree) was plucked from the bark. Male and female samples were kept separate in sealed bags. Each sample weighed approximately 1 g. The sealed samples were stored in a freezer at −20° C. for no longer than one week until extraction.

### Extraction

The samples, already separated into four sets (May males, May females, August males, and August females) were taken out of the freezer and placed into four corresponding pre-weighed 500 mL Erlenmeyer flasks. Field identification of males and females was verified for accuracy. The flasks containing the thawed plant matter were reweighed to determine the total mass of dwarf mistletoe in each flask. Approximately 300 mL of 98.8% methanol was added to each flask. The flasks were sealed and left for approximately 8 hours after which the resulting extract was drained off into four correspondingly labeled 2 L Erlenmeyer flasks. These flasks were similarly sealed to prevent contamination and only opened during the addition of a further 3×300 mL aliquots of methanol extract creating a total volume of approximately 1200 mL in each flask.

The 4×2 L Erlenmeyer flasks, each containing the four individual aliquots of methanol extraction, then underwent rotary evaporation at 45° C. in round-bottom flasks until the extract thickened to the consistency of a paste. After evaporation, each resulting thick extract was diluted again with 10 mL methanol, sonicated, and transferred to 20 mL vials. Once each extract was in its respective vial, filtered air was used to evaporate the methanol overnight, and then placed into the vacuum chamber of a high-pressure vacuum to remove any water residue. Approximately 20% (w/w) of solid dry “raw” extract was extracted from the initial mass of dried shoots. These extracts were stored in a dark cupboard until subsequent testing.

### Preparation of Media for Growth of Test Organisms

Mueller-Hinton media was prepared from a combination of Mueller-Hinton broth and agar. Four flasks of media were prepared so that four representative bacteria could be cultured and measured (1% bacterial volume/150 mL media). Once the media mixture was made, it was autoclaved with an exposure time of 30 minutes at 121° C. The media was then placed into a hot water bath set at approximately 57° C. to maintain the media in liquid form until inoculation and plating.

### Preparation of Test Organisms

Four strains of medically important bacteria were chosen to test the extracts, namely: Staphylococcus aureus (Gram-positive), methicillin-resistant Staphylococcus aureus (MRSA) (Gram-positive), Salmonella enterica (Gram-negative), and Escherichia coli (Gram-negative). Cultures of each bacteria were initially streaked for isolated colonies by plating from stock cultures onto blood agar plates and incubated at 35° C. for 24 hours. From these plates, isolated colonies were then chosen and re-plated to verify the purity of the cultures. Once the cultures were grown and purity was verified, they were stored in the fridge until needed for testing. Prior to plating for extract testing, each strain was grown in test tubes containing 2 mL sterile Mueller-Hinton broth, loosely capped and placed in a shaker incubator at 35° C. until they reached the 0.5 McFarland Turbidity Standard.

### Extract Preparation

Prior to plating the media, each solid dry “raw” extract was re-suspended in methanol to obtain a 1:3 dilution (1 part dry extract: 3 parts methanol). The tubes containing the suspensions were then vigorously shaken manually and vortexed to ensure thorough mixing. The extracts were capped and stored in the fridge.

### Inoculation and Plating

Once the bacteria reached 0.5 McFarland Turbidity Standard, 1.5 mL of each type of bacteria was added to each flask containing Mueller-Hinton media and swirled to ensure even distribution. Each flask was then immediately plated into 2 sterile large Petri dishes labeled with the corresponding bacteria. The plates were then left to dry to prevent bacterial “swimming”. Once the plates were set, they were moved into a laminar flow hood for further drying.

### Antimicrobial Assays—Plates

The modified Kirby Bauer Disc Diffusion Method was used to determine antimicrobial activity. 6 mm discs were soaked in one of the four extract suspensions for approximately 1 minute and dried in the laminar flow hood. Each plate was then inoculated with all 4 extracts (one per disc), one negative control disc, and one positive control disc. The positive control for the Gram-positive bacteria was erythromycin and for the Gram-negative bacteria was gentamicin. The negative control was methanol. Once the discs were all in place the plates were incubated at 35° C. for 24 hours. Assays were replicated and repeated.

### Results—Antimicrobial Assays—Plates

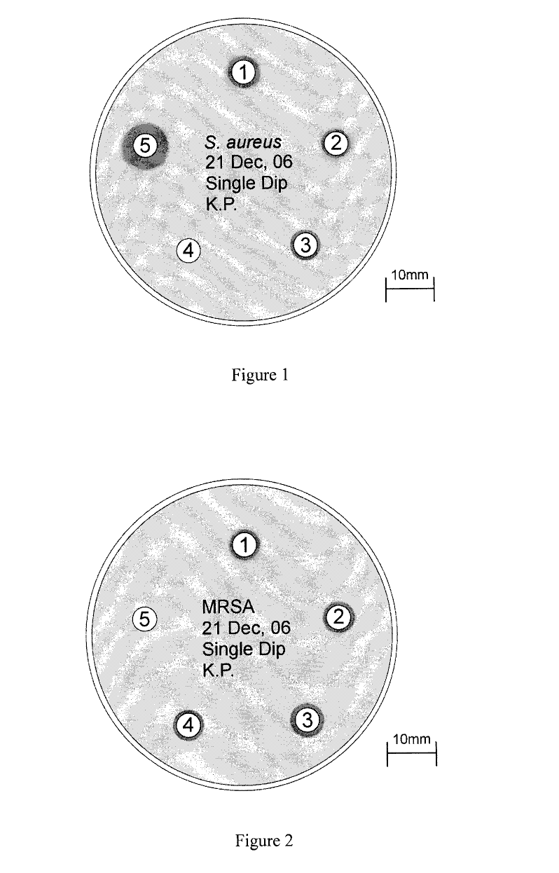

The results from the disc diffusion assays are summarized in Table 1. Typical results with Staphylococcus aureus and MRSA are shown in FIGS. 1 and 2. The zones of inhibition for the Gram-positive samples Staphylococcus aureus and MRSA are considerably large. It is noted that the extract inhibits the growth of MRSA whereas the positive control does not. Gram-negative samples Salmonella and Escherichia coli showed no observable zones of inhibition other than that with the positive control. The negative control showed no zone of inhibition (results not shown).

TABLE 1
Summary of results obtained from antimicrobial testing of “raw” A. americanum extracts.

| | Extract #1 | Extract #2 | Extract #3 | Extract #4 | Positive |
|---|---|---|---|---|---|
| | May Males | May Females | August Males | August Females | Control |
| Bacterial | Zone of | Zone of | Zone of | Zone of | Zone of |
| plate | inhibition (mm) | inhibition (mm) | inhibition (mm) | inhibition (mm) | inhibition (mm) |
| S. aureus | 9 ± 1 | 9 ± 1 | 10 ± 1 | None | 22 ± 1 |
| MRSA | 8 ± 1 | 9 ± 1 | 12 ± 1 | 7 ± 1 | None |
| E. coli | None | None | None | None | 15 ± 2 |
| Salmonella | None | None | None | None | 25 ± 1 |

### Antimicrobial Assays—Optical Density

Optical density (OD) measurements were taken to determine how each extract was affecting the growth pattern of each type of bacteria. Only Extracts #1 and #3 (May males and August males, respectively, at an initial concentration of 1 part dry extract: 3 parts methanol) were used for OD measurements, due to the strong antimicrobial activity they exhibited in the plate assays. The OD measurements were conducted by adding 40 mL aliquots of Mueller-Hinton broth into 17 flasks and varying the methanol and/or extract concentrations. One flask was a negative control (methanol), 2 flasks were positive controls (same antibiotics used in plate assays), and 2 flasks were standards for the growth curve. The flasks containing bacteria were inoculated at a 1% volume/volume ratio and incubated in shakers at 35° C. over a 10 hour period. The OD was measured at a wavelength of 600 nm at 30 minute intervals.

### Results—Antimicrobial Assays—Optical Density

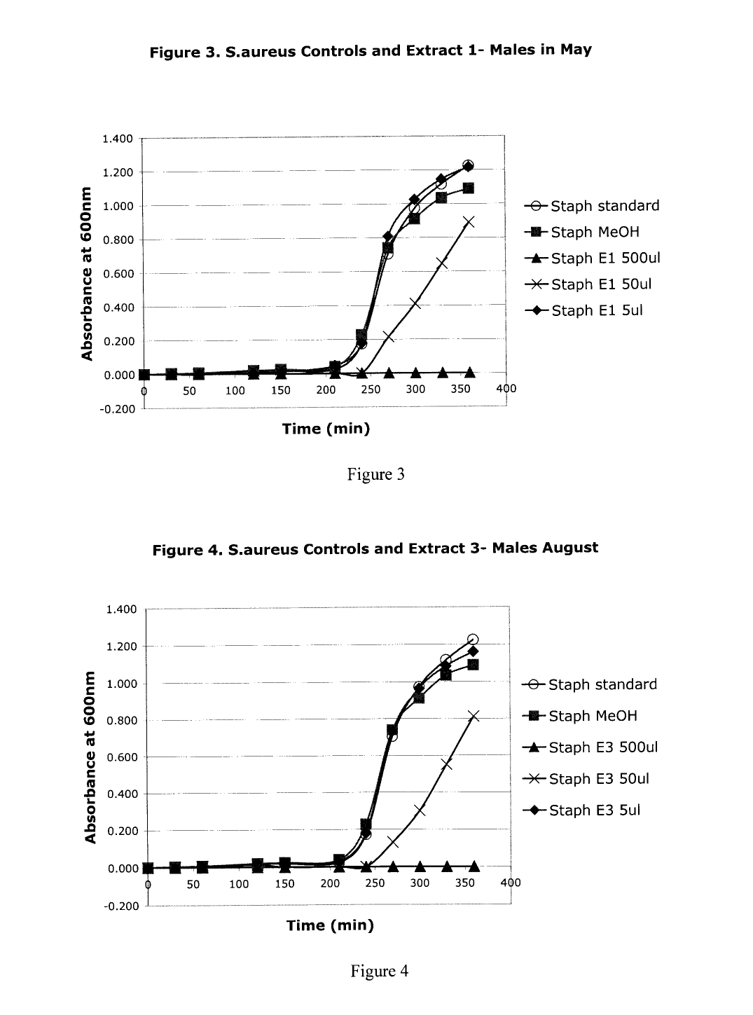

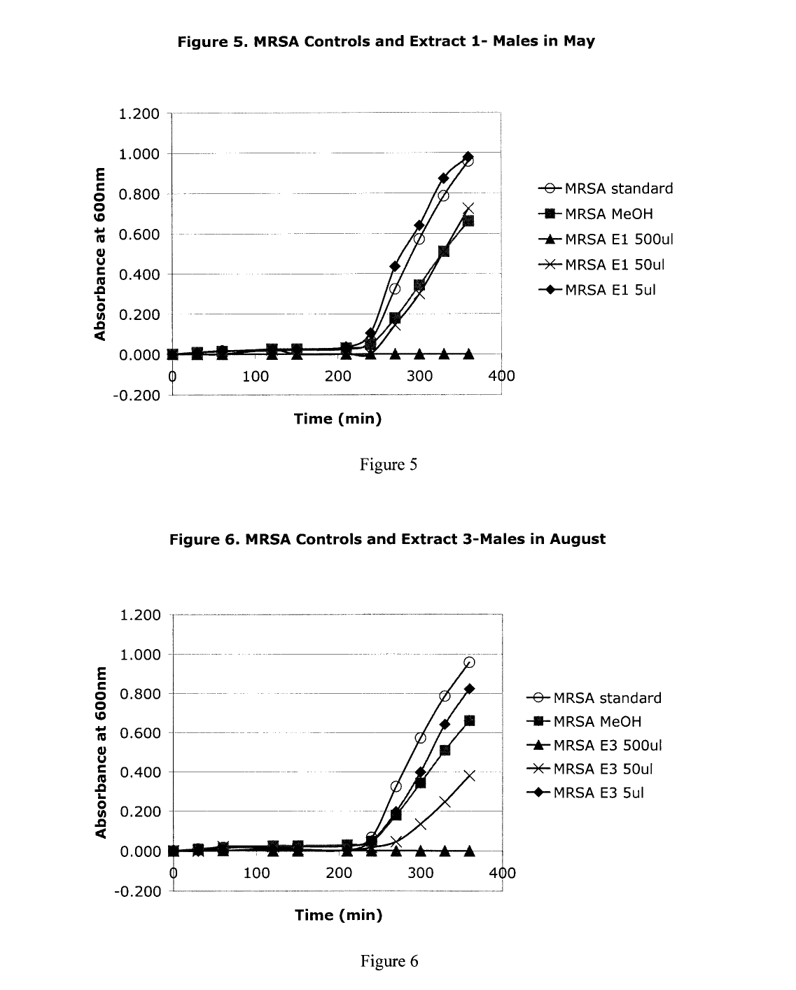

The results of the OD assays are shown in FIGS. 3 to 6. The slope of the OD graphs (growth versus time) provides an indication of bacterial growth rate. For both Extracts #1 and #3 the highest concentration (500μL) was effective in preventing growth. The 50 μL concentration, although showing signs of bacterial growth, also showed inhibition of growth when compared to the standard curve. The 5 μL sample showed little, if no, detectable changes in the growth pattern when compared to the standard curve. The methanol negative control had no visible effects on bacterial growth.

### Example 2

### Sample Collection and Extraction

Male Arceuthobium americanum shoots were collected in August and solid dry “raw” extract was obtained therefrom according to the protocol set out in Example 1.

### Purification of Raw Extract

A 9:1 methanol/water mix (about 200 mL) was used to dissolve about 3 g of the “raw” extract. The mix was then placed in a separatory funnel with an equal volume of hexanes. The funnel was then agitated and the mixture was allowed to separate into its two components. The separation was repeated two more times with fresh hexanes to complete an efficient extraction. The repetition and use of fresh hexanes ensured an effective removal of compounds more soluble in hexanes (those that are less polar). Both the 9:1 methanol/water mix fraction and the hexanes fraction were rotary evaporated to dryness to obtain two dried extracts, the “9:1” extract and the “hexanes” extract, respectively. The “9:1” extract represented about 1% (w/w) of the initial mass of dried shoots.

### Purification of “9:1” Extract

The “ 9:1” extract was dissolved in a 6:4 methanol/water mixture (200 mL), combined with an equal volume chloroform in a separatory funnel and agitated. The resulting emulsion was allowed to separate, and, following removal and concentration of the chloroform portion using rotary evaporation, was extracted again with fresh chloroform. The repetition and fresh chloroform served to sufficiently and efficiently separate the mixture by pulling substances more soluble in chloroform (due to relative polarity) from the methanol/water mixture. The methanol/water mixture was then rotary evaporated to dryness to obtain a dried extract, the “6:4” extract. The “6:4” fraction represented about 0.5% w/w of the initial mass of dried shoots.

### Extract Preparation and Antimicrobial Assays—Plates

The “raw” extract, “9:1” extract, “hexanes” extract, and the “6:4” extract were re-suspended test tubes containing methanol to obtain a working concentration of 0.1 mg/μL. The tubes were then vigorously shaken and vortexed to ensure thorough mixing. The extracts were capped and refridgerated until they were bioassayed according to the disc diffusion assay protocols set out in Example 1 against the following medically important bacterial strains: Staphylococcus aureus (Gram-positive), methicillin-resistant Staphylococcus aureus (MRSA, ATCC 43300) (Gram-positive), vancomycin-resistant Enterococcus faecalis (VRE, ATCC 51299) (Gram-positive), a Bacillus sp. (Gram-positive), and Salmonella typhi (Gram-negative), and Escherichia coli (Gram-negative).

### Results—Antimicrobial Assays—Plates

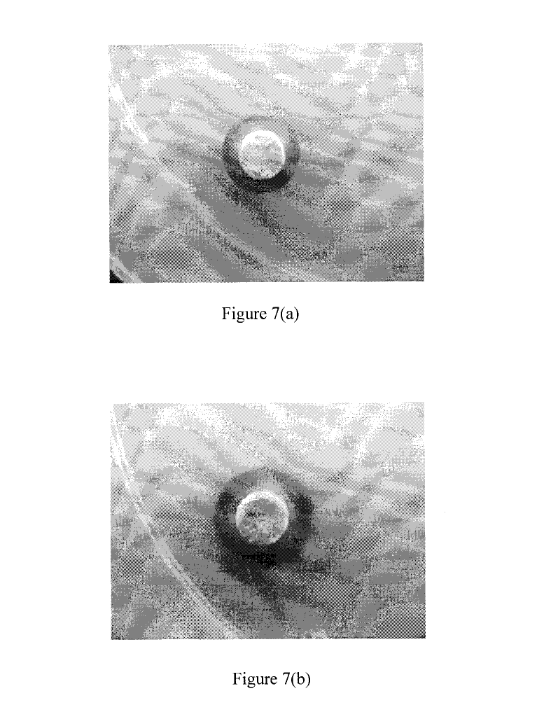

Results from the disc diffusion assay of the “raw” extract on each kind of bacteria are summarized in Table 2. The zones of inhibition for the Gram-positive samples S. aureus, MRSA, and Bacillus sp. are considerably large; as in Example 1, the extract inhibited the growth of MRSA, whereas the positive control, erythromycin, did not. The zone of inhibition for MRSA was consistently larger than that of its non-resistant counterpart, S. aureus; typical results of the “6:4” extract with Staphylococcus aureus and MRSA are shown in FIG. 7. VRE was not susceptible. The zones of inhibition for the gram-negative samples Salmonella and E. coli, as in Example, 1 showed no observable zones of inhibition other than that of the positive control. The negative control showed no zone of inhibition (not shown).

TABLE 2
Summary of results obtained from antimicrobial testing
of “raw” A. americanum extracts.

| | Zone of inhibition (mm) of | Zone of inhibition (mm) of |
|---|---|---|
| Bacteria | raw extract | positive control |
| S. aureus | 9 ± 1 | 22 ± 1 |
| MRSA | 11 ± 1 | None |
| VRE | None | None |
| Bacillus sp. | 9 ± 1 | 20 ± 1 |
| E. coli | None | 15 ± 2 |
| Salmonella | None | 25 ± 1 |

Each disc had effectively 3 mg dried raw extract from an application of 30 μL of a 1 mg/μL concentration (n = 3 discs, and trials were repeated three times). Negative control data (methanol) not shown.

Results also indicated that the antimicrobial component(s) of the “raw” extract partitioned into the more polar fractions in the subsequent separation steps (Table 3). In the initial separation, the activity was found in the 9:1 methanol/water fraction rather than the hexanes fraction. In the second step, the activity was in the 6:4 methanol/water fraction rather than the chloroform fraction. These findings are surprising as many organic antimicrobial compounds have a greater affinity for chloroform than the more polar 6:4 methanol/water. The size of the zone of inhibition did not appear to change when the dosage was increased from 30 μL to 50 μL, which suggests that 30 μL of a 1 mg/μL application is approaching the limit of diffusion.

TABLE 3
Summary of results obtained from antimicrobial testing of
various fractions of A. americanum extract against S. aureus
with 20 μL, 30 μL and 50 μL doses of 1 mg/μL extracts
(n = 3 discs, trials were repeated three times).

| | Zone of inhibition | Zone of inhibition | Zone of inhibition |
|---|---|---|---|
| | (mm) with 20 μL | (mm) with 30 μL | (mm) with 50 μL |
| Fraction | dose | dose | dose |
| raw | 8 ± 1 | 9 ± 1 | 9 ± 1 |
| 9:1 | 8 ± 1 | 9 ± 1 | 9 ± 1 |
| hexanes | 0 | 0 | 0 |
| 6:4 | 8 ± 1 | 8 ± 1 | 8 ± 1 |
| chloroform | 0 | 0 | 0 |

Control data not shown.

MRSA displayed zones of inhibition when grown with discs containing extract from the “raw”, the “9:1” and the “6:4” fractions (Table 4). The zones of inhibition were similar for these fractions, and consistently larger than the zones for S. aureus. FIG. 7 shows the zone of inhibition of the “6:4” extract and S. aureus compared to the same extract and MRSA.

TABLE 4
Summary of results obtained from antimicrobial testing of
various fractions of A. americanum extract against MRSA.

| | Zone of inhibition |
|---|---|
| Fraction | (mm) on MRSA |
| raw | 11 ± 1 |
| 9:1 | 11 ± 1 |
| 6:4 | 11 ± 1 |

Each disc had effectively 3 mg dried “raw” extract from an application of 30 μL of a 1 mg/μL concentration (n = 3 discs, and trials were repeated three times). Control data not shown.

As will be apparent to those skilled in the art in the light of the foregoing disclosure, many alterations and modifications are possible in the practice of this invention without departing from the spirit or scope thereof.

1. An antibiotic composition comprising an extract from a plant belonging to the genus Arceuthobium.

2. A composition according to claim 1, wherein the extract is extracted from Arceuthobium americanum.

3. A composition according to claim 2, wherein the extract is extracted from male plants.

4. A composition according to claim 3, wherein the extract is extracted from shoots of the male plants.

5. A composition according to claim 4, wherein the shoots of the male plants are harvested in the late spring or summer.

6-7. (canceled)

8. A pharmaceutical composition comprising a therapeutically effective amount of the composition of claim 1 combined with at least one pharmaceutically acceptable diluent, carrier or adjuvant.

9. A pharmaceutical composition according to claim 8 for the treatment of an infection caused by a gram-positive bacteria.

10. A pharmaceutical composition according to claim 8 for the treatment of an infection caused by a drug-resistant bacteria.

11. A pharmaceutical composition according to claim 10 wherein the drug-resistant bacteria is methicillin-resistant Staphylococcus aureus.

12. A pharmaceutical composition according to claim 8 wherein the pharmaceutical composition is in the form of a capsule, tablet, powder, liquid or gel.

13. A pharmaceutical composition according to claim 8 wherein the pharmaceutical composition is provided in a form suitable for oral, nasal, ophthal, rectal, vaginal, topical, intravenous, subcutaneous, or parenteral administration.

14-15. (canceled)

16. A method of treating or preventing a bacterial infection, the method comprising administering to a patient a composition comprising a therapeutically effective amount of an extract from a plant belonging to the genus Arceuthobium.

17. A method according to claim 16, wherein the extract is extracted from Arceuthobium americanum.

18. A method according to claim 17, wherein the extract is extracted from male plants.

19. A method according to claim 18, wherein the extract is extracted from shoots of the male plants.

20. A method according to claim 19, wherein the shoots of the male plants are harvested in the late spring or summer.

21. A method according to claim 16 wherein the bacterial infection is caused by gram-positive bacteria.

22. A method according to claim 16 wherein the bacterial infection is an infection caused by an antibiotic-resistant bacteria.

23. A method according to claim 22 wherein the antibiotic-resistant bacteria is methicillin-resistant Staphylococcus aureus.

24. A method according to claim 16 wherein the composition is administered by oral, nasal, ophthal, rectal, vaginal, topical, intravenous, subcutaneous, or parenteral administration.

25-40. (canceled)
